(19) United States
(12) Patent Application Publication
(10) Pub. No.: US 2020/0214945 A1
(43) Pub. Date: Jul. 9, 2020

(54) SKIN-BRIGHTENING COSMETIC MASK SYSTEM

(71) Applicant: L'OREAL, Paris (FR)

(72) Inventor: Anne-Laure Suzanne BERNARD, New York, NY (US)

(21) Appl. No.: 16/431,332

(22) Filed: Jun. 4, 2019

Related U.S. Application Data
(63) Continuation-in-part of application No. 16/239,422, filed on Jan. 3, 2019.

(51) Int. Cl. A61K 8/02; A61K 8/22; A61K 8/73; A61K 8/44; A61K 8/365; A61K 8/31; A61Q 19/02

(52) U.S. Cl. CPC A61K 8/0212; A61K 8/22; A61K 8/733; A61K 8/44; A61K 2800/884; A61K 8/31; A61Q 19/02; A61K 2800/882; A61K 8/365

(57) ABSTRACT

A system for forming a cosmetic mask that can deliver oxygen and skin brightening includes a first composition that includes at least one oxidizer and at least one cross-linkable polymer; and a second composition that includes at least one alkaline booster; and at least one catalyst. Contacting each of the first and second compositions together on a substrate results in cross-linking the cross-linkable polymer, whereby a continuous film is formed and simultaneously oxygen is released and contained within the film.

### RELATED APPLICATIONS

This application is related to United States patent application entitled CATALYTIC SYSTEMS INCLUDING A PEROXIDE FOR DELIVERY OF OXYGEN TO SKIN AND RELATED METHODS OF USE, Ser. No. 16/239,422, filed Jan. 3, 2019, which is incorporated herein, in its entirety.

### FIELD OF TECHNOLOGY

The present disclosure is directed to oxygenate skin treatment systems which include compositions that are combined to deliver oxygenate skin treatment. More specifically, the present disclosure is directed to oxygenate skin treatment systems that provide a continuous film formed on a skin substrate, which in some examples is used for enhancing oxygenate skin treatment, the systems including catalytic activator of film and oxygen formation.

### BACKGROUND

Oxygen therapy has drawn significant attention in recent years for uses such as skin-brightening, skin repair, and wound healing. Lack of oxygen in wounded areas may result in poor collagen deposition and a prolonged healing process. Oxygen, of course, is present in air and is taken into the human body via respiration, and such respiratory oxygen must dissolve into body fluid to circulate into tissues to support cellular activities. Supplemental oxygen may be delivered from the external environment via diffusion through the skin barrier (hyperbaric oxygen therapy), however, due to the strong barrier function of skin and the conversion of oxygen from the gaseous to dissolved state, the delivery efficiency is relatively low. Additional challenges arise for hyperbaric oxygen therapy, in that some patients cannot tolerate the side effects of the treatments, afford the treatments, or gain access to a hyperbaric chamber.

Topical delivery of dissolved oxygen is another approach. Dissolved oxygen may be delivered to a skin surface via the use of perfluorocarbons as oxygen carriers or through chemical degradation of peroxide compounds to generate oxygen gas. A burst treatment of supplemental oxygen may inhibit proliferation of anaerobic bacteria, accelerate fibroplasia and epithelialization. Typically, such products employ oxidizing agents such as hydrogen peroxide. However, contact with hydrogen peroxide in such products may, under certain conditions, produce mild to moderate skin irritation and eye tearing.

U.S. Patent Application Publication No. 2014/0294944 A1 to Schumacher discloses a composition for the delivery of oxygen having microencapsulated peroxide and microencapsulated catalyst that liberate oxygen upon sufficient contact with each. However, accomplishing the microencapsulation of hydrogen peroxide as disclosed in this publication is both difficult and complicated.

U.S. Pat. No. 9,181,093 B2 to Karandikar et al., discloses a two-part spray for the liberation and delivery of oxygen through the use of a first part that is a peroxide-containing solution and a second part that is a nanoparticle manganese dioxide catalyst, which, when mixed together, provide oxygen. However, the manganese dioxide nanoparticles utilized in this publication are not water-soluble which complicates delivery of the composition. Moreover, due to the encapsulation, the reaction efficiency would well be sacrificed, and the oxygen release could be impeded. Further, there may be respiratory health concerns with encapsulated particles.

It is an object of the present invention to provide an oxygenate skin treatment system that overcomes at least one of the aforementioned drawbacks, wherein the system employs oxidizers, for example, peroxides, to achieve oxygen release and skin brightening benefits and is free of one or more of ammonia and persulfates.

In addition to the above-mentioned challenges associated with delivery of oxygen treatment, there are limited systems that enable containment of oxygen or other brightening skin treatment systems. Thus, it is a further object of the present invention to provide an oxygenate skin treatment system in that provides a film, or mask, that is water based, hydrating and enhances delivery of skin brightening treatments, including those that employ oxygen generation, and is easily removable.

### BRIEF SUMMARY OF THE INVENTION

This summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In accordance with various embodiments, provided is a system for forming a cosmetic mask, comprising:
a) a first composition comprising:
   i) at least one oxidizer (peroxide); and
   ii) at least one cross-linkable polymer;
b) a second composition comprising:
   i) at least one alkaline booster;
   ii) at least one catalyst; and
      wherein contacting each of the first and second compositions together on a substrate results in cross-linking the cross-linkable polymer, whereby a continuous film is formed and simultaneously oxygen is released and contained within the film.

In accordance with some embodiments, one or both of the first and second compositions are aqueous.

In accordance with some embodiments, the at least one oxidizer comprises at least one peroxide selected from one or a combination of hydrogen peroxide, PVP peroxide, urea peroxide, carbamide peroxide, calcium peroxide, magnesium peroxide, barium peroxide, zinc peroxide, lithium peroxide, dibenzoyl peroxide, peracetic acid, and sodium peracetate.

In accordance with some embodiments, the at least one cross-linkable polymer comprises at least one alginate.

In accordance with some embodiments, the at least one alkaline booster is selected from one or a combination of guanidine carbonate and arginine.

In accordance with some embodiments, the at least one cross-linking catalyst is selected from one or a combination of manganese gluconate, manganese pyrrolidone carboxylic acid, zinc gluconate, zinc pyrrolidone carboxylic acid, calcium chloride, calcium pyrrolidone carboxylic acid, and calcium gluconate.

In accordance with some embodiments, one or both of the compositions comprises at least one fatty compound and wherein the at least one fatty compound is selected from one or a combination of mineral oil, hem isqualane, dimethicone, squalane, castor oil, isohexadecane, and coconut oil.

In accordance with some embodiments, one or more of the at least one oxidizer is present in the first composition from about 2% by weight, based on the total weight of the composition; the at least one cross-linkable polymer is present in the first composition from about 1% by weight, based on the total weight of the composition; the at least one fatty compound is present in the first composition from at least about 20% by weight, based on the total weight of the composition; the at least one alkaline booster is present in the second composition from about 2% by weight, based on the total weight of the composition; the at least one catalyst is present in the second composition from about 0.3% by weight, based on the total weight of the composition.

In accordance with some embodiments, one or both of the compositions comprises at least one fatty compound, and wherein the at least one fatty compound is present in the second composition from at least about 30% by weight, based on the total weight of the composition.

In other embodiments, provided is a method of forming an occlusive cosmetic mask for providing oxygenate brightening to skin, comprising:
a) applying to a region of skin a first composition comprising a combination of at least one oxidizer, and at least one cross-linkable polymer;
b) applying to the region of skin a second composition comprising, at least one alkaline booster, and at least one catalyst
wherein one or both of the compositions optionally comprises at least one fatty compound.

In accordance with some embodiments, the steps of the method include (a) and (b) in any order.

In accordance with some embodiments, each of the compositions is aqueous and is applied in liquid form.

In accordance with some embodiments, one or both of the compositions is applied using a spray applicator.

In accordance with some embodiments, the compositions are preblended before applying to the region of skin.

In other embodiments, provided is an article of manufacture for providing a skin brightening system in an occlusive continuous film, comprising: at least first and second separately contained compositions, the brightening system comprising hydrogen peroxide; at least one cross-linkable polymer; at least one alkaline booster; at least one catalyst; and optionally at least one fatty compound which when present is from about at least 30% by weight, based upon the total weight of the composition, wherein the skin-brightening cosmetic system is ammonia-free and persulfate-free:
a) the first composition comprising the hydrogen peroxide, and the cross-linkable polymer, and
b) the second composition comprising the alkaline booster, and the catalyst
wherein either one or both of the first and second compositions comprises one or more of the optional fatty compounds, if present, and optionally one or more of solvents, and optionally one or more actives and other components.

In accordance with some embodiments, the first and second compositions are one of (i) contained to be mixable prior to application to skin, and (ii) contained to be applied separately, in any order.

In another aspect, the present disclosure provides system generally including a skincare formulation comprising a peroxide; and an applicator including a peroxide-decomposing catalyst, wherein the peroxide-decomposing catalyst is configured to decompose the peroxide to provide oxygen gas.

In accordance with any of the embodiments disclosed herein, the peroxide-decomposing catalyst maybe coupled to an application surface of the applicator. In accordance with any of the embodiments disclosed herein, the application surface may be a surface of a mask configured to conform to a face of a wearer. In accordance with any of the embodiments disclosed herein, the application surface may be selected from the group consisting of a bristle surface of a brush, a roller surface of a rolling applicator, and a blade surface of a spatula. In accordance with any of the embodiments disclosed herein, the skincare formulation may be disposed in a first compartment of a sachet and the application surface may be a surface of a second compartment of the sachet, and wherein the first compartment and the second compartment are fluidically separated by a breakable barrier.

In accordance with any of the embodiments disclosed herein, the peroxide-decomposing catalyst may coupled to the application surface with a binder. In accordance with any of the embodiments disclosed herein, the binder may be selected from the group consisting of latexes, polyacrylates, elastomers, hydrogels, and combinations thereof.

In accordance with any of the embodiments disclosed herein, the applicator includes a crosslinked hydrogel including the peroxide catalyst dispersed therein. In accordance with any of the embodiments disclosed herein, the crosslinked hydrogel may be freestanding. In accordance with any of the embodiments disclosed herein, the crosslinked hydrogel may be in a shape of a mask configured to conform to a face of the user. In accordance with any of the embodiments disclosed herein, the crosslinked hydrogel may be in a shape of one or more bristles of a brush.

In accordance with any of the embodiments disclosed herein, the peroxide-decomposing catalyst may be selected from the group consisting of iron oxide and a transition metal-based catalyst; an iron salt; sodium tungstate dehydrate, phosphoric acid, and a phase-transfer catalyst comprising a quaternary ammonium hydrogen sulfate; manganese dioxide; sodium bicarbonate and manganese dioxide; and a manganese salts.

In accordance with any of the embodiments disclosed herein, the skincare formulation further comprises an additive selected from the group consisting of a clay, an alkaline agent, a humectant, an antioxidant, and combinations thereof.

In accordance with any of the embodiments disclosed herein, the peroxide may be selected from the group consisting of hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof.

In accordance with any of the embodiments disclosed herein, a concentration of the peroxide in the skincare formulation may be between about 0.1 wt. % and about 4 wt. %.

In accordance with any of the embodiments disclosed herein, the skincare formulation may be selected from the group consisting of an emulsion, a paste, a gel, and combinations thereof.

In another aspect, the present disclosure provides a method of contacting a portion of skin with oxygen gas generally including contacting a skincare formulation including a peroxide with an applicator including a peroxide-decomposing catalyst, thereby generating oxygen gas in the skincare formulation; and contacting the portion of skin with the skincare formulation with the applicator. In accordance with any of the embodiments disclosed herein, the peroxide-decomposing catalyst may be coupled to an application surface of the applicator, and wherein contacting the skincare formulation with the peroxide-decomposing catalyst includes contacting the skincare formulation with the application surface of the applicator.

In accordance with any of the embodiments disclosed herein, an oxygen concentration in the skincare formulation 1 minute after contacting the skincare formulation with the peroxide-decomposing catalyst may be in a range of about 15 mg/L to about 100 mg/L.

In accordance with any of the embodiments disclosed herein, contacting the portion of skin with the skincare formulation may include contacting the portion of skin with the skincare formulation for a duration of between about 1 minute and about 20 minutes.

Other features and advantages of the present invention will be apparent from the following more detailed description, taken in conjunction with the accompanying drawings which illustrate, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### BRIEF DESCRIPTION OF THE DRAWINGS

FIG. 1 is a plan view of a mask applicator, in accordance with an embodiment of the disclosure.

FIG. 2 is a perspective view of a system including another applicator, in accordance with an embodiment of the disclosure.

FIG. 3 is a perspective view of another system including yet another applicator, in accordance with an embodiment of the disclosure.

FIG. 4 is a perspective view of another applicator, in accordance with an embodiment of the disclosure.

FIG. 5 is a perspective view of an applicator, in accordance with an embodiment of the disclosure.

FIG. 6A is a top-down plan view of a system, in accordance with an embodiment of the disclosure.

FIG. 6B is a perspective view of the system of FIG. 6A.

FIG. 7 schematically illustrates a method, in accordance with an embodiment of the disclosure.

Aspects and many of the attendant advantages of the claimed subject matter will become more readily appreciated as the same become better understood by reference to the following detailed description, when taken in conjunction with the accompanying drawings.

The detailed description set forth below in connection with the appended drawings, where like numerals reference like elements, is intended as a description of various embodiments of the disclosed subject matter and is not intended to represent the only embodiments. The embodiments described in this disclosure are provided merely as examples or illustrations and should not be construed as preferred or advantageous over other embodiments. The illustrative examples provided herein are not intended to be exhaustive or to limit the claimed subject matter to the exact forms disclosed.

### DETAILED DESCRIPTION OF THE INVENTION

The term “alkaline booster” as used herein means compositions comprising at least one alkaline agent with a final pH>7, or a final pH>8. The alkaline agent may be organic or mineral or hybrid with a pKa at 25° C. greater than 7.5.

The terms “ammonia-free” and “persulfate-free” means that the composition comprises less than a trace amount, for example, less than 0.001% by weight of the composition, of an ammonia containing ingredient or a persulfate containing ingredient, and more particularly that the ingredient has not been intentionally added, but may be included as a by-product or carry-over of another ingredient. In some embodiments; each of ammonia-free and persulfate-free means that the composition is devoid, respectively, of ammonia and ammonia containing ingredients, and of persulfates, persulfate containing ingredients, perborates, alkali metal percarbonates, alkaline-earth metal percarbonates, peracids and precursors thereof.

“Continuous film” means and refers to a film having an essentially uninterrupted surface along the length and breadth of the film as applied to a substrate, for example, a keratinous substrate such as skin. In some embodiments, the film has a thickness that varies across a surface on which it is spread, such as but not limited, to application on all or portions of facial skin. In addition to other features, the film can contain the oxygen on or adjacent the skin surface to enhance the efficacy of oxygen benefits. Further, the film can prevent migration of irritating hydrogen peroxide vapor into the eyes, thus minimizing irritation to the eyes. In another embodiment, the continuous film includes an essentially homogenous composition with an essentially uniform thickness. In some embodiments, the thickness of the film does not vary by more than 5%.

“Cosmetically acceptable” means compatible with any keratinous substrate. For example, “cosmetically acceptable carrier” means a carrier that is compatible with any keratinous substrate.

The terms “occlusion” and “occlusive” as used herein refer to the property of being at least partially vapor proof such that a film or mask formed according to the disclosure is effective at retaining oxygen and other vapors between the film and the substrate.

The term “oxidation resistant” as used in the context of a fatty compound means and refers to a fatty compound, such as an oil, that is free of unsaturated functions, ester groups. The fatty compound can be chosen from C6-C19 lower alkanes, non-silicone oils of animal, plant, mineral or synthetic origin fatty alcohols, fatty acids, non-silicone waxes, and silicones.

“Skin-brightening” means and refers to improvement in radiance, glow effect and lightening effect to keratinous tissue. Skin brightness is related to luminosity and can be measured instrumentally by the change of luminosity (ΔL).

Provided herein are systems, compositions and methods for providing a cosmetic mask that employ a crosslinkable polymer in first part (or first composition) and divalent metal salts in a second part (or second composition), wherein the two parts are mixed to provide crosslinked polymer that can be applied to a substrate such as skin to form a film. Also provided are compositions and methods that include peroxide in a first part and divalent metal salts in a second part, wherein the two parts are mixed to generate oxygen. In certain embodiments, as further described herein below, the crosslinkable polymer and peroxide are combined in a single first part, and the divalent salts are present in the second part, wherein the two parts are mixed to provide crosslinked polymer that can be applied to a substrate such as skin to form a film and which can generate oxygen that is retained within the film on the surface of the substrate. In some embodiments, the systems compositions and methods include providing a cosmetic mask for brightening keratinous tissue, such as skin. In some embodiments, a system according to the disclosure is provided as a two part skin care product that includes in a first part, or composition, a cross-linkable polymer and oxygen generating actives, for example oxidizers such as peroxides, and includes in a second part, or composition, one or more catalysts, particularly oxidizer decomposing catalysts and alkaline boosters. In the various embodiments, one or more of the compositions optionally includes at least one fatty compound. In some embodiments, one or both of the first and second compositions includes a fatty compound.

In some particular embodiments, a system according to the disclosure is provided as a two part skin care product that includes in a first part, or composition, a cross-linkable polymer and oxygen generating actives, for example oxidizers such as peroxides, and at least one fatty compound, and includes in a second part, or composition, one or more catalysts, particularly oxidizer decomposing catalysts, and one or more agents for brightening including boosters for oxygen generation and one or more fatty compounds.

According to the methods, each of the first and second compositions are contacted on a substrate to form a film, in some embodiments a continuous film. In some particular embodiments, essentially simultaneously with the film formation, the compositions as combined also release oxygen which is retained beneath the film and between the film and the substrate. In some embodiments, the film is peelable for removal. In use, the crosslinked film formed during application can improve brightening efficacy via occlusion and improve skin comfort by prolonging oxygen retention on the skin surface and preventing vapor release to minimize tearing.

Applicants have found that the systems unexpectedly provide the film together with the brightening agents in the absence of ammonia and persulfate containing alkaline boosters to achieve brightening. The system provides a film that, when combined with an oxygen generating system as described herein, demonstrates skin-brightening efficacy and provides a skin smoothing effect to impart a glow to the skin. The compositions are ammonia-free and persulfate-free and demonstrate efficiency in whitening that is at least the same as with ammonia containing compositions.

Oxidizer

In accordance with the various embodiments, the systems and compositions include at least one oxidizer. In some embodiments, the oxidizer is selected from peroxides that are compatible with cosmetic applications. In the various embodiments, the at least one peroxide may be selected from hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof. In some embodiments, the composition may comprise PVP peroxide (hydrogen peroxide and PVP polymer complexes). In some particular embodiments, the peroxide includes hydrogen peroxide.

In accordance with the various embodiments, the amount of oxidizer present in a composition according to the disclosure can range from about 0.1% to about 4%, or from about 0.2% to about 3.0%, or from about 0.5 to about 2%, or from about 1% to about 1.5%, or from about 2.0% to about 2.5%, or from about 2.5% to about 3.0%, or from about 3.0% to about 3.5%, or from about 3.5% to about 4.0%, or any suitable combination, sub-combination, range, or sub-range thereof by weight, based on the weight of the composition. One of ordinary skill in the art, however, will appreciate that other ranges are within the scope of the invention.

Thus, any one of or a combination of oxidizers may be present, by weight, based on the total weight of the composition, or from about 0.5, 0.6, 0.7, 0.8, 0.9, 1.0, 1.5, 2.0, 2.5, 3.0, 3.5, to about 4 weight percent, including increments and ranges therein and there between.

Cross-Linkable Polymer

In accordance with the various embodiments, the systems and compositions include at least one cross-linkable polymer. In some embodiments, the at least one cross-linkable polymer is selected from alginates. In some particular embodiments, the cross-linkable polymer includes at least one alginate. Alginates are copolymers with blocks of (1-4)-linked β-D-mannuronate (M) and its C-5 epimer α-L-guluronate (G) residues. The content of the M blocks and G blocks may be described by the M/G ratio of the alginate. The plurality of sodium alginates may include any suitable average M/G ratios, including, but not limited to an M/G ratio between 70:30 to 50:50, alternatively between 70:30 to 60:40, alternatively between 65:35 to 55:45, alternatively between 60:40 to 50:50, or any suitable combination, sub-combination, range, or sub-range thereof by weight. The at least one alginate may include any suitable average molecular weight, including, but not limited to, between 20 kDa to 425 kDa, or any suitable combination, sub-combination, range, or sub-range thereof. In some examples, the at least one alginate may include: an average molecular weight, including, but not limited to, of about 375-425 kDA, and percentage of (1-4)-linked β-D-mannuronate (M) of about 65-70% M; an average molecular weight, including, but not limited to, of about 300-400 kDa, and percentage of (1-4)-linked β-D-mannuronate (M) of about 50% M; an average molecular weight, including, but not limited to, of about 90-180 kDA, and percentage of (1-4)-linked β-D-mannuronate (M) of about 60-70% M; an average molecular weight, including, but not limited to, of about 20-60 kDA, and percentage of (1-4)-linked β-D-mannuronate (M) of about 25-35% M; and combinations of these.

In accordance with the various embodiments, the amount of cross-linkable polymer present in the systems and compositions can range from about 0.5% to about 5%, or from about 0.5% to about 1.0%, or from about 1.0 to about 1.5%, or from about 1.5% to about 2.0%, or from about 2.0% to about 2.5%, or from about 2.5% to about 3.0%, or from about 3.0% to about 3.5%, or from about 3.5% to about 4.0%, or from about 4.0% to about 4.5%, or from about 4.5% to about 5.0%, or any suitable combination, sub-combination, range, or sub-range thereof by weight, based on the weight of the composition. One of ordinary skill in the art, however, will appreciate that other ranges are within the scope of the invention.

Thus, any one of or a combination of cross-linkable polymers may be present, by weight, based on the total weight of the composition, or from about 0.5, 0.6, 0.7, 0.8, 0.9, 1.0, 1.5, 2.0, 2.5, 3.0, 3.5, 4.0, 4.5, to about 5 weight percent, including increments and ranges therein and there between.

Catalyst

In accordance with the various embodiments, the systems and compositions include at least one catalyst that includes at least one water-soluble oxidizer decomposition catalyst. In some embodiments the catalyst is a cross-linking catalyst that includes at least one water-soluble oxidizer decomposition catalyst. In particular, the catalyst may be any suitable peroxide decomposition catalyst, including, but not limited to, a metal salt. Suitable metal salts include, but are not limited to, a manganese salt of pyrrolidone carboxylic acid, manganese gluconate, manganese sulfate, manganese chloride, a copper salt of pyrrolidone carboxylic acid, copper gluconate, copper sulfate, copper chloride, a zinc salt of pyrrolidone carboxylic acid, zinc gluconate, zinc sulfate, zinc chloride, or combinations thereof. In some particular embodiments, the catalyst includes one or more of manganese gluconate, manganese pyrrolidone carboxylic acid, zinc gluconate, zinc pyrrolidone carboxylic acid, calcium chloride, calcium pyrrolidone carboxylic acid, and calcium gluconate.

In some particular embodiments, the catalyst for crosslinking polymer is selected from magnesium salts, calcium sulfate, calcium carbonate, calcium pantothenate, and combinations thereof. In some particular embodiments, the catalyst for oxygen generation is selected from alkali metal silicates, for example, sodium silicate, lithium silicate, magnesium silicate, potassium silicate and combinations thereof. In accordance with the various embodiments, the amount of the at least one catalyst present in a composition according to the disclosure can range from about 0.1% to about 6.0%, or from about 0.5% to about 5.5%, or from about 1% to about 5%, or from about 1.5% to about 4.5%, or from about 0.5% to about 1.0%, or from about 1.0% to about 1.5%, or from about 1.5% to about 2.0%, or from about 2.0% to about 2.5%, or from about 2.5% to about 3.0%, or from about 3.0% to about 3.5%, or from about 3.5% to about 4.0%, or from about 4.0% to about 4.5%, or from about 4.5% to about 5.0%, or from about 5.0% to about 5.5%, or from about 5.5% to about 6.0%, or from about 6.0% to about 6.5%, or any suitable combination, sub-combination, range, or sub-range thereof by weight, based on the weight of the skin treatment system. One of ordinary skill in the art, however, will appreciate that other ranges are within the scope of the invention.

Thus, any one of or a combination of catalysts may be present, by weight, based on the total weight of the composition, or from about from about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0 to about 6.5 weight percent, including increments and ranges therein and there between.

Alkaline Booster

In accordance with the various embodiments, the systems and compositions include at least one or more ammonia-free, persulfate-free alkaline boosters. In some embodiments, the alkaline booster is selected from organic amines and salts, mineral based salts, alkali metal hydroxides, alkali earth metal hydroxides, alkali metal salts, and combinations thereof. In some embodiments, the alkaline booster is selected from guanidine carbonate, arginine, monoethanolamine, triethanolamine, potassium hydroxide, sodium bicarbonate and combinations thereof. In some embodiments the composition includes with the alkaline booster an optional catalyst. In some embodiments the optional catalyst is manganese dioxide. Although these alkaline boosters are given as an example, it will be appreciated that other alkaline boosters compatible with cosmetic applications known in the art may be used. Thus, the alkaline booster may be organic or mineral or hybrid with a pKa at 25° C. greater than 7.5. For example, lysine, carnosine, KOH, NaOH, and the like.

Non-limiting examples of alkaline agents include alkali metal hydroxides (e.g., lithium hydroxide, sodium hydroxide, potassium hydroxide, magnesium hydroxide), alkaline-earth metal hydroxides (e.g., calcium hydroxide), alkali metal silicates, (e.g., sodium silicate, lithium silicate, and potassium silicate), alkali metal carbonates (e.g., lithium carbonate, sodium carbonate, potassium carbonate), alkaline-earth metal carbonates (e.g., calcium carbonate), organic carbonates (e.g., guanidine carbonate), basic amino acids (arginine, lysine, histidine), and their polymers (poly arginine, poly lysine, etc.), organic amines, such as alkanolamines (e.g., monoethanolamine, diethanolamine, triethanolamine, aminomethyl propanol), ammonium hydroxide, and a mixture thereof.

Organic amines include, but are not limited to, those having one or two primary, secondary, or tertiary amine functions, and at least one linear or branched C1-C8 alkyl groups bearing at least one hydroxyl radical. Organic amines include cyclic amines and other cyclic compounds, saturated or unsaturated, having one or more nitrogen atoms within the ring, and mixtures thereof. The organic amines may be chosen from those having a pKb at 25° C. of less than 12, such as less than 10 or such as less than 6. It should be noted that this is the pKb corresponding to the function of highest basicity.

Organic amines may also be chosen from alkanolamines such as mono-, di- or trialkanolamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof.

Non-limiting examples of alkanolamines include monoethanolamine (also known as monoethanolamine or MEA), diethanolamine, triethanolamine, monoisopropanolamine, aminomethylpropoanol, diisopropanolamine, triisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol (aminomethyl propanol or AMP), tris(hydroxymethylamino)methane, tetrahydroxypropyl ethylenediamine, tromethamine, a mixture thereof, preferably triethanolamine and/or aminomethyl propanol. Other examples include but are not limited to: 1,3-diaminopropane, 1,3-diamino-2-propanol, spermine, and spermidine.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form. Polymeric forms are also useful, such as poly arginine, poly lysine, etc. Additional non-limiting examples include basic amino acids comprising an additional amine function optionally included in a ring or in a ureido function. In some cases, amino acids that may be used include, but are not limited to, aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine.

In instances, the organic amines may be organic amines of heterocyclic type. Besides histidine that has already been mentioned in the amino acids, non-limiting mention may also be made of pyridine, piperidine, imidazole, 1,2,4-triazole, tetrazole, and benzimidazole. In some cases, the organic amines may be amino acid dipeptides. Amino acid dipeptides that may be used in the present disclosure include but not limited to carnosine, anserine, and baleine.

The organic amines may also be chosen from compounds comprising a guanidine function. Organic amines of this type include, besides arginine that has already been mentioned as an amino acid, creatine, creatinine, 1,1-dimethylguanidine, 1,1-diethylguanidine, glycocyamine, metformin, agmatine, N-amidinoalanine, 3-guanidinopropionic acid, 4-guanidinobutyric acid, and 2-([amino(imino)methyl]amino)ethane-1-sulfonic acid.

The organic amine may be in salt form. The term “organic amine salt,” as used herein, means organic or mineral salts of an organic amine as described above. As a non-limiting example, the organic salts may be chosen from the salts of organic acids, such as citrates, lactates, glycolates, gluconates, acetates, propionates, fumarates, oxalates and tartrates. In one embodiment, the organic salt is trisodium citrate. Further as a non-limiting example, the mineral salts may be chosen from hydrohalides (for example hydrochlorides), carbonates, hydrogen carbonates, sulfates, hydrogen phosphates, and phosphates. The ammonium salts that may be used according to the present disclosure may be chosen from the following acid salts: carbonate, bicarbonate. For instance, the salt is the carbonate, such as ammonium carbonate.

The alkali metal phosphates and carbonates that may be used are, for example, sodium phosphate, potassium phosphate, sodium carbonate, sodium bicarbonate, potassium carbonate, potassium bicarbonate, and their derivatives.

Alkaline agents may also be chosen from inorganic bases and hydroxide bases such as alkali metal hydroxides, alkaline-earth metal hydroxides, transition metal hydroxides, quaternary ammonium hydroxides, organic hydroxides, and mixtures thereof. Suitable examples are ammonium hydroxide, sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and mixtures thereof.

In some instances, the at least one alkaline agent is chosen from aminomethyl propanol, sodium hydroxide, potassium hydroxide, lithium hydroxide, aminomethyl propanediol, triisopropanol amine, dimethylstearylamine, dimethyl/tallowamine, lysine, ornithine, arginine, monoethanolamine, triethanolamine, calcium hydroxide, calcium bicarbonate, and mixtures thereof. Furthermore, in some instances, the at least one alkaline agent is chosen from aminomethyl propanol, sodium hydroxide, lithium hydroxide, calcium hydroxide, monoethanolamine, triethanolamine, trisodiuim citrate, and mixtures thereof. Moreover, in some instances, the at least one alkaline agent is chosen from an alkanolamine, for example, an alkanolamine selected from monoethanolamine, diethanolamine, triethanolamine, aminomethyl propanol, tris(hydroxymethyl)aminomethane, tetra hydroxy propyl ethylenediamine, a mixture thereof, preferably triethanolamine, aminomethyl propanol, and a mixture thereof.

In some particular embodiments, alkaline boosters include one or a combination of guanidine carbonate and arginine.

In accordance with the various embodiments, the amount of alkaline booster present in a composition according to the disclosure can range from about 0.1% to about 10%, or from about 0.5% to about 7%, or from about 1.5% to about 4.5%, or from about 0.5% to about 1.0%, or from about 1.0% to about 1.5%, or from about 1.5% to about 2.0%, or from about 2.0% to about 2.5%, or from about 2.5% to about 3.0%, or from about 3.0% to about 3.5%, or from about 3.5% to about 4.0%, or from about 4.0% to about 4.5%, or from about 4.5% to about 5.0%, or from about 5.0% to about 5.5%, or any suitable combination, sub-combination, range, or sub-range thereof by weight, based on the weight of the composition. In some embodiments according to the disclosure, the composition includes at least about 2% of the at least one alkaline booster. Thus, in some embodiments, the at least one alkaline booster is present in an amount that is not less than about 2%, based upon the total weight of the composition. One of ordinary skill in the art, however, will appreciate that other ranges are within the scope of the invention. In some embodiments, two or more alkaline boosters are present.

Thus, any one of or a combination of alkaline boosters may be present, by weight, based on the total weight of the composition, or from about 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, to about 10.0 weight percent, including increments and ranges therein and there between.

Fatty Compound

In accordance with the disclosure, one or more fatty compound or oil may be present in the composition. The fatty compound includes one or more of non-silicone oils of animal; plant; animal or synthetic origin, fatty alcohols, fatty acids; esters of a fatty acid and/or of a fatty alcohol, non-silicone waxes, and silicones.

In some embodiments, the fatty compound includes, but is not limited to: 1) C6-C19 lower alkanes, non-silicone oils of animal, plant, animal or synthetic origin, fatty alcohols, fatty acids, non-silicone waxes, and silicones; 2) hydrocarbon-based oils of animal origin, such as perhydrosqualene; 3) fluoro oils, perfluoromethycyclopentane and perfluoro-1,3-dimethylcyclohexane, sold under the name FLUTEC® PC1 and FLUTEC® PC3 by the company BNFL Fluorochemicals; perfluoro-1,2-dimethylcyclobutane, perfluoroalkanes such as dodecafluoropentane and tetradecafluorohexane, sold under the names PF 5050® by the company 3M, or bromoperfluorooctyl sold under the name FORALKYL® by the company Atochem; nonafluoromethoxybutane and nonafluoroethoxyisobutane; perfluoromorpholine derivatives such as 4-trifluoromethyl perfluoromorpholine sold under the same PF 5052® by the company 3M. 4) linear or branched saturated fatty alcohols having from 6 to 30 carbon atoms or from 8 to 30 carbon atoms, for instance cetyl alcohol, stearyl alcohol, and the mixture thereof (cetylstearyl alcohol), octyldodecanol, 2-butyloctanol, 2-hexyldecanol, 2-undecylpentadecanol, oleyl alcohol, and linoleyl alcohol. In some particular embodiments, a composition according to the disclosure includes one or more of mineral oil, hemisqualane, dimethicone, squalane, castor oil, isohexadecane, and coconut oil.

In some embodiments, at least one fatty substance describes an organic compound that is insoluble in water at ordinary ambient temperature (25° C.) and at atmospheric pressure (760 mmHg). In some embodiments, the at least one fatty substance has a water solubility of less than 5%. In some embodiments, the at least one fatty substance has a water solubility of less than 1%. In some embodiments, the at least one fatty substance has a water solubility of less than 0.1%. Although these fatty compounds are given as an example, it will be appreciated that other compounds compatible with cosmetic applications known in the art may be used.

In accordance with the various embodiments, the amount of fatty compound present in a composition according to the disclosure can range from about 0% to about 60%, or from about 35% to about 60%, or from about 40% to about 59%, or from about 45% to about 58%, or from about 50% to about 57%, or from about 52% to about 56%, or from about 30% to about 55%, or from about 40% to about 60% or any suitable combination, sub-combination, range, or sub-range thereof by weight, based on the weight of the composition. In some embodiments according to the disclosure, the composition includes at least about 30% of the fatty compound. And, in some embodiments according to the disclosure, the composition includes at least about 50% of the fatty compound. Thus, in some embodiments, the fatty compound is present in an amount that is not less than about 30%, or not less than about 50%, based upon the total weight of the composition. One of ordinary skill in the art, however, will appreciate that other ranges are within the scope of the invention. In some embodiments, two or more fatty compounds are present. And, in some embodiments, one or more fatty compounds are present wherein at least one is an oxidation resistant fatty compound.

Thus, any one of or a combination of fatty compounds, if present, may be present, by weight, based on the total weight of the composition, or from about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to about 60 weight percent, including increments and ranges therein and there between.

Solvent

In accordance with the disclosure, one or more solvent is present in the composition. The solvent present in the cosmetic composition, according to the disclosure, includes, but is not limited to, water, alcohol, propylene glycol, or combinations thereof. In some embodiments, the solvent includes only water. In other embodiments, the solvent includes water and one or more other solvents. Although these solvents are given as an example, it will be appreciated that other solvents compatible with cosmetic applications known in the art may be used. In some particular embodiments, solvents include water.

In accordance with the various embodiments, solvent is present in a given composition in an amount of from about 1% to about 70%, or any suitable combination, sub-combination, range, or sub-range thereof by weight, based on the weight of the composition. One of ordinary skill in the art, however, will appreciate that other ranges are within the scope of the invention.

Thus, one or a combination of solvents may be present, by weight, based on the total weight of the composition from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, to about 70 weight percent, including increments and ranges therein and there between.

Water

The compositions comprise from about 1% to about 70% by weight of water, with respect to the total weight of the composition. In some embodiments, the amount of water in the composition can range from about 1% to about 50%, or from about 10% to about 50%, or from about 10% to about 20%, or from about 15% to about 25%, or from about 20% to about 30%, or from about 25% to about 35%, or from about 30% to about 40%, or from about 35% to about 45%, or from about 40% to about 50%, or any suitable combination, sub-combination, range, or sub-range thereof by weight, based on the weight of the composition.

The pH of the composition is not limited but is generally between 2 and 12, and in some embodiments is one of between 3 and 11, and between 5 and 9, and between 6 and 8, and 7. In accordance with the various embodiments, the pH of the first part is in the range from about 3.0 to about 4.5, and the pH of the second part is in the range from about 8 to about 12. The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid. More particularly the pH of Part A (hydrogen peroxide containing) part is adjusted by phosphoric acid; the pH of Part B (alkaline booster containing) is tuned by concentration of alkaline agents.

Thus, water may be present by weight, based on the total weight of the composition, or from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, to about 70 weight percent, including increments and ranges therein and there between.

Optional Components

In some embodiments, there may be one or more actives present in the cosmetic composition, according to the disclosure, the additive selected from, for example, humectants, such as acetamide MEA, glycols, such as glycerin and propylene glycol; alcohol; anti-microbial components, salicylic acid, alpha hydroxy acid, including, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid; and vitamins and vitamin derivatives, such as calcium pantothenate, tocopherol and ascorbic acid; and combinations thereof.

In some embodiments, there may be one or more other components present in the cosmetic composition, according to the disclosure, the other components selected from, fillers such as clays, talc, organic thickeners with for instance, anionic, cationic, nonionic, and amphoteric polymeric associative thickeners and combinations thereof; penetrants; sequestrants; fragrances; dispersants; film-forming agents; ceramides; opacifiers and combinations thereof. Although the aforementioned optional components are given as an example, it will be appreciated that other optional components compatible with cosmetic applications known in the art may be used.

In accordance with the various embodiments, amount of actives and other components present in the composition can range from about 0 to about 50%, or from about 0.5 to about 30%, or from about 1.5 to about 20%, and from about 5 to about 15%, or any suitable combination, sub-combination, range, or sub-range thereof by weight, based on the weight of the composition.

In some embodiments, one or more actives, alone or in combination, can be present in the composition according to the disclosure from about 0.05 to about 50% by weight, or from about 0.05% to about 2.5% by weight, or from about 0.1 to about 2%, or from about 0.25 to about 1.5%, and from about 0.5 to about 1.25%, or any suitable combination, sub-combination, range, or sub-range thereof by weight, based on the weight of the composition.

In some embodiments, one or more other components, such as preservatives, vitamins, and the like, alone or in combination, can be present in the composition according to the disclosure from about 0.05 to about 50% by weight, or from about 0.05% to about 25% by weight, or from about 0.1 to about 10%, or from about 0.25% to about 5%, and from about 0.5 to about 3.5%, or any suitable combination, sub-combination, range, or sub-range thereof by weight, based on the weight of the composition. In some exemplary embodiments, preservatives may include sodium salicylate, and vitamins may include ascorbic acid, tocopherol and combinations of these.

Thus, one or a combination of optional components may be present, by weight, based on the total weight of the composition, or from about 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

Articles of Manufacture

In accordance with the various embodiments, the composition may be provided in a kit or other article of manufacture.

It will be appreciated that in the various embodiments of packaging, that the sub compositions that are packaged are maintained as separate until the composition is intended to be applied to keratinous tissue, at which time they are combined and mixed. Generally, the oxidizing material is separate from the catalyst/alkaline booster components until the composition is intended to be applied to keratinous tissue. Thus, one sub composition includes at least the hydrogen peroxide and any additional optional oxidizing agent, and one sub composition includes at least the alkaline booster. Variously, each of the sub compositions may include any one or more of the fatty compounds and any one or more of solvents, surfactants, polymers, actives and other components. Further, any one or more of the fatty compounds and any one or more of solvents, actives and other components may be separately packaged from the other sub compositions, such that the article of manufacture may comprise more than two separate packages or package chambers.

In one embodiment, the article of manufacture may be a packet with separate packages or chambers separated by a frangible seal between the chambers. In use, the seal is broken by the user to contact the separately packaged sub compositions and mix them prior to application onto the skin. In yet other embodiments, the article of manufacture may be a tube with dual chambers. Two parts are mixed when squeezing out from each chamber. In yet other embodiments, the article of manufacture may be a dual-chamber pump and the two parts can be mixed after pumping out of the container. In some other embodiments, the packaging is a single tube with a seal between the two components that is broken to mix. In other embodiments, the packaging can be a single container holding a suspension of encapsulated material that is mixed/broken to disperse and mix. And in yet other embodiments, the packaging is otherwise sufficient to retain the oxidizing material separate from the catalyst/alkaline booster components until the composition is intended to be applied to keratinous tissue.

In some embodiments, the one or both of the first and second parts may be delivered by spray application, or other applicator selected from a pump, or brush.

In accordance with the various embodiments, a composition of the system for forming a continuous film mask is in a form including a suspension, lotion, cream, serum, essence, gel, stick, spray, ointment, paste, foam, mousse, cream, wipe, patch, strip, film-forming product.

### Examples

Raw Materials:

Compositions and systems as described in the representative embodiments herein are selected from commercially available materials, including, for example: hydrogen peroxide, mineral oil, hemisqualane, dimethicone, squalane, castor oil, isohexadecane, coconut oil, align, manganese gluconate, manganese pyrrolidone carboxylic acid, arginine, guanidine carbonate, zinc gluconate, zinc pyrrolidone carboxylic acid, calcium chloride, calcium pyrrolidone carboxylic acid, calcium gluconate, calcium pantothenate, low-ester pectin.

Inventive Compositions and Systems

In some embodiments, the system includes at least two parts wherein a first part, Part A, includes: hydrogen peroxide; one or more of mineral oil, hemisqualane, dimethicone, squalane, castor oil, isohexadecane, and coconut oil; and align, and wherein a second part, Part B, includes: one or more of mineral oil, hem isqualane, dimethicone, squalane, castor oil, isohexadecane, and coconut oil; one or more of manganese gluconate, manganese pyrrolidone carboxylic acid, zinc gluconate, zinc pyrrolidone carboxylic acid, calcium chloride, calcium pyrrolidone carboxylic acid, and calcium gluconate; one or more of arginine and guanidine carbonate; and optionally one or more additional additive for example one of calcium pantothenate and low-ester pectin. Two exemplary embodiments of the inventive compositions that include brightening and film compositions are shown in Table 1.

TABLE 1
Inventive Brightening Compositions

| | Inventive | Inventive |
|---|---|---|
| | Example 1 | Example 2 |
| INCI INGREDIENT | (wt %) | (wt %) |
| Manganese PCA | 0.5 | |
| Tetrasodium Etidronate | 0.03 | 0.03 |
| EDTA | 0.1 | 0.1 |
| Manganese | | 0.15 |
| Gluconate | | |
| Arginine | 2 | 2 |
| Hydrogen | 2 | 2 |
| Peroxide | | |
| Tetrasodium Pyrophosphate | 0.02 | 0.02 |
| Water | 34.1175 | 34.4675 |
| Mineral Oil | 56.75 | 56.75 |
| Surfactants | 3.53 | 3.53 |
| Optional Actives | 0.1775 | 0.1775 |
| Polymer | 0.775 | 0.775 |

Various system compositions were prepared according to the disclosure, the first and second parts including film components and brightening components.

TABLE 2
Inventive system compositions, with film and varied catalyst

| | Inventive Example 3 | | | Inventive Example 4 | | |
|---|---|---|---|---|---|---|
| | (with oil, Mn Glu as the catalyst) | | | (without oil, Mn PCA as the catalyst) | | |
| | | | Final (A + B, | | | Final (A + B, |
| Type | Part A | Part B | Equal Parts) | Part A | Part B | Equal Parts) |
| Oxidizer | 4 | | 2 | 4 | | 2 |
| (Hydrogen Peroxide) | | | | | | |
| Hydrogen Peroxide | 0.3 | | 0.15 | 0.3 | | 0.15 |
| Stabilizers | | | | | | |
| Cross Linkable | 2 | | 1 | 3 | | 1.5 |
| Polymer | | | | | | |
| Manganese | | 0.6 | 0.3 | | | |
| Gluconate | | | | | | |
| Manganese | | | | | 1 | 0.5 |
| Pyrrolidone | | | | | | |
| Carboxylic Acid | | | | | | |
| Calcium Chloride | | 5 | 2.5 | | 5 | 2.5 |
| Booster (Arginine) | | 4 | 2 | | 4 | 2 |
| Squalane | 30 | 50 | 40 | | | |
| Surfactants | 5 | 3 | 4 | | | |
| Thickener | | 0.6 | 0.3 | | 1 | 0.5 |
| Others | 0.5 | | 0.25 | 0.5 | | 0.25 |
| Water | QS 100 | QS 100 | QS 100 | QS 100 | QS 100 | QS 100 |

TABLE 3
Inventive system compositions, with film and without film

| | Inventive Example 5 | | | Inventive Example 6 | | |
|---|---|---|---|---|---|---|
| | (with oil, and film) | | | (with oil, without film) | | |
| | | | Final (A + B, | | | Final (A + B, |
| Type | Part A | Part B | Equal Parts) | Part A | Part B | Equal Parts) |
| Oxidizer | 4 | | 2 | 4 | | 2 |
| (Hydrogen Peroxide) | | | | | | |
| Hydrogen Peroxide | 0.3 | | 0.15 | 0.3 | | 0.15 |
| Stabilizers | | | | | | |
| Cross Linkable | 2 | | 1 | | | |
| Polymer | | | | | | |
| Calcium Chloride | | 5 | 2.5 | | | |
| Booster (Arginine) | | 4 | 2 | | 4 | 2 |
| Squalane | 30 | 50 | 40 | 30 | 50 | 40 |
| Surfactants | 5 | 3 | 4 | 5 | 3 | 4 |
| Thickener | | 0.6 | 0.3 | 1 | 0.6 | 0.8 |
| Others | 0.5 | | 0.25 | 0.5 | | 0.25 |
| Water | QS 100 | QS 100 | QS 100 | QS 100 | QS 100 | QS 100 |

Testing

1. In Vivo Evaluation of Eye Irritation

Two volunteers applied two formulas on face (half face with inventive example 5 and the other half with inventive example 6). After five minutes, the side with comparative example 2 shows more eye irritation than the side with comparative examples 1.

2. In Vitro Stratum Corneum Brightening Evaluation

In vitro stratum corneum tests were conducted as follows: three stratum corneum samples (pre-conditioned for 17 h at 75% relative humidity) were treated with compositions for an exposure period of 15 minutes with no external heat or occlusion. Following treatment, the samples were rinsed and dried. The color of the stratum corneum was measured before and after treatment, and the individual change of Luminosity L* before and after treatment on white background was calculated and reported as ΔL (change in luminosity).

| | Inventive Example 5 | Comparative Example 6 |
|---|---|---|
| Type | (with oil, and film) | (with oil, without film) |
| ΔL | 2.33 ± 0.17 | 2.91 ± 0.20 |

Catalytic Systems; Articles

The following discussion provides examples of systems and methods suitable for contacting a portion of skin with oxygen gas. Several examples discussed below include systems including skincare formulation comprising hydrogen peroxide and a hydrogen peroxide-decomposing catalyst. As discussed in greater detail herein, such hydrogen peroxide-decomposing catalysts are configured to decompose hydrogen peroxide to generate oxygen gas. When the skincare formulation is applied to portion of skin, for example, with the surface including the hydrogen peroxide-decomposing catalyst the portion of skin is exposed to heightened levels of oxygen.

Oxygen therapy including exposing skin to oxygen is believed to have therapeutic benefits including, for example, enhanced wound healing, skin brightening, skin detoxification, and skin repair. Conventional methods of oxygen delivery, such as compressed oxygen gas or exposing hydrogen peroxide to alkaline environments, generally result in low levels of oxygen that generate oxygen slowly. Such low levels of oxygen may be insufficient to provide certain benefits associated with providing oxygen therapy to skin and wounds.

Toward that end, in certain aspects the present disclosure provides systems and methods for providing oxygen to a portion of skin. In certain embodiments, the systems of the present disclosure include a skincare formulation comprising a peroxide; and an applicator including a peroxide-decomposing catalyst. As discussed further herein, the peroxide-decomposing catalyst is configured to decompose the peroxide to provide oxygen gas. In this regard, by contacting the skincare formulation including a peroxide with the applicator including a peroxide-decomposing catalyst oxygen gas is generated and is dissolved or otherwise dispersed in the skincare formulation. The portion of skin may be contacted with the skincare formulation containing oxygen, such as with the applicator, thereby delivering high levels of oxygen to the portion of skin.

In the following description, numerous specific details are set forth in order to provide a thorough understanding of one or more embodiments of the present disclosure. It will be apparent to one skilled in the art, however, that many embodiments of the present disclosure may be practiced without some or all of the specific details. Further, it will be appreciated that embodiments of the present disclosure may employ any combination of features described herein.

In an aspect, the present disclosure provides a system for delivery of oxygen to a portion of skin. In an embodiment, the system includes a skincare formulation comprising a peroxide; and an applicator including a peroxide-decomposing catalyst, wherein the peroxide-decomposing catalyst is configured to decompose the peroxide to provide oxygen gas. In an embodiment, the applicators of the present disclosure are provided separately from the skincare formulations described herein.

In an embodiment, the peroxide-decomposing catalyst is coupled to an application surface of the applicator. In an embodiment, the peroxide-decomposing catalyst is coupled to the application surface with a binder. In this regard, the peroxide-decomposing catalyst is on the application surface and configured to contact the skincare formulation and to apply skincare formulation that has contacted the peroxide-decomposing catalyst. In an embodiment, the binder is selected from the group consisting of latexes, polyacrylates, elastomers, hydrogels, and combinations thereof. In an embodiment, the application surface includes a plurality of fibers coated with the peroxide-decomposing catalyst. In an embodiment, the plurality of fibers are cast or otherwise formed into a non-woven fabric, such as in the shape of an applicator.

In an embodiment, the applicator includes a gel, such as a crosslinked hydrogel, and the peroxide-decomposing catalyst is disposed within the gel. Such gels may be prepared by, for example, crosslinking a mixture including gel precursors and the peroxide-decomposing catalyst such that the peroxide-decomposing catalyst is disposed therein. By crosslinking the gel, the peroxide-decomposing catalyst is fixed within the crosslinked matrix. In this regard, the gel including the peroxide-decomposing catalyst is configured to decompose peroxide contained in skincare formulations described herein to provide oxygen gas when such skincare formulations are contacted with the gel. In an embodiment, the gel has sufficient structural integrity, such as through a sufficiently high level of crosslinking, that it is free-standing. In an embodiment, the gel has sufficient structural integrity that the applicator including the gel is not coupled to a separate structure, such as a structure to provide additional rigidity to the applicator.

By coupling the peroxide-decomposing catalyst to the applicator, such as by coupling the peroxide-decomposing catalyst to an application surface and/or fixing the peroxide-decomposing catalyst in a crosslinked gel, the applicators of the present disclosure may be used two or more times to decompose peroxide in a skincare formulation. In this regard, the applicators of the present disclosure and the skincare formulations of the present disclosure may be sold separately from one another. Further, by using a heterogeneous peroxide-decomposing catalyst coupled to an application surface and/or fixed in a crosslinked gel, a user may selectively contact a skincare formulation with the peroxide-decomposing catalyst. This is in contrast to skincare formulations in which a peroxide-decomposing catalyst is dissolved or dispersed, such as on particles dispersed therein. In such a configuration, peroxide in the skincare formulation is depleted, such as during shipment and storage, and, consequently, oxygen is also generated and depleted constantly, thus ultimately providing less oxygen to a user.

In an embodiment, the applicator is a single-layer or single-sheet applicator on which the peroxide-composing catalyst is fixedly coupled. This is in contrast to a system including two or more layers or sheets that are movably independent of one another.

The systems of the present disclosure include a peroxide-decomposing catalyst. The peroxide-decomposing catalyst can be configured to decompose peroxide in a skincare formulation to provide oxygen dispersed or dissolved therein. In an embodiment, the peroxide-decomposing catalyst includes iron oxide and a transition metal-based catalyst. In certain further embodiments, the peroxide-decomposing catalyst further includes an iron salt. In an embodiment, the iron salt is selected from the group consisting of ferrous sulfate, ferrous gluconate, ferric chloride, and combinations thereof. In an embodiment, the peroxide-decomposing catalyst includes sodium tungstate dehydrate, phosphoric acid, and a phase-transfer catalyst comprising a quaternary ammonium hydrogen sulfate. In an embodiment, the peroxide-decomposing catalyst includes manganese dioxide. In an embodiment, the peroxide-decomposing catalyst includes sodium bicarbonate, manganese dioxide, and a manganese salt. In an embodiment, the manganese salt is selected from the group consisting of manganese gluconate, manganese PCA, manganese chloride, and combinations thereof.

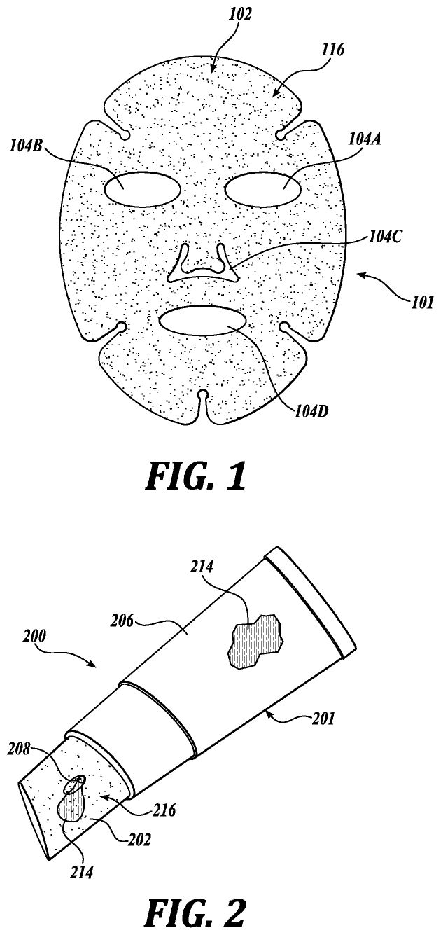

Turning now to FIG. 1 there is shown an example of an applicator 101 in accordance with embodiments of the present disclosure. In the illustrated embodiment, applicator 101 is in the form of a mask configured to conform to the face of a user and includes apertures 104A-104D, application surface 102, and peroxide-decomposing catalyst 116. In this regard, a skincare formulation (not shown, see FIGS. 2, 3, and 6) as described herein may be contacted with application surface 102, thereby decomposing peroxide in the skincare formulation to provide oxygen. The skincare formulation now containing oxygen dissolved or otherwise dispersed therein may be applied to, for example, a face of a user with the mask 101 thereby contacting the skin of the face with oxygen. As discussed elsewhere herein, in an embodiment, the peroxide-decomposing catalyst 116 is coupled to application surface 102 such as with a binder. In other embodiments, the mask 101 comprises a gel including peroxide-decomposing catalyst 116 dispersed therein and, for example, does not include a surface to which the gel is coupled.

In an embodiment, the systems of the present disclosure include an applicator having an application surface in the form of a slider 200, for example, a spatula. In that regard, attention is directed to FIG. 2 in which a system 200 in accordance with an embodiment of the disclosure is illustrated. As shown, system 200 includes applicator 201 having an applicator surface 202 including a peroxide-decomposing catalyst 216. In an embodiment, skincare formulation 214 is disposed in a portion of the applicator 201. In the illustrated embodiment, applicator surface 202 further includes an aperture 208 through which skincare formulation 214 disposed in handle 206 may be extruded, such as through application of pressure to handle 206 by a user. In this regard, a user may contact the skincare formulation 214 with the catalytic application surface 202, thereby generating oxygen in the skincare formulation 214, before applying the skincare formulation 214 to a portion of skin.

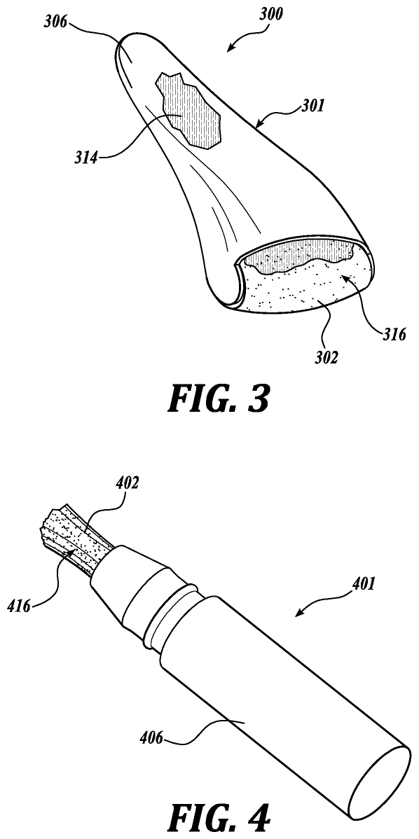

In another embodiment, the systems of the present disclosure include an applicator in the form of a rolling applicator and the application surface is a rolling application surface. In that regard, attention is directed to FIG. 3 in which system 300 including rolling applicator 301 in accordance with an embodiment of the disclosure is illustrated. Rolling applicator 301 is shown to include a handle 306 and rolling applicator surface 302 including a peroxide-decomposing catalyst 316. Rolling applicator surface 302 is configured to roll, such as when rolling applicator surface 302 is applied to a portion of skin. In that regard, a user may contact rolling applicator surface 302 with a skincare formulation 314 as described further herein and apply the skincare formulation 314 to a portion of skin, for example, after a duration sufficient to generate oxygen in the skincare formulation 314. In the illustrated embodiment, skincare formulation 314 is disposed in a portion of handle 306 such that it may be placed in contact with the rolling applicator surface 302, such as by a user applying pressure to handle 306.

In an embodiment, the applicators of the present disclosure are in the form of a brush and the application surface includes a surface of a plurality of bristles. In that regard, attention is directed to FIG. 4 in which an applicator brush 401 in accordance with embodiments of the present disclosure is illustrated. As shown, applicator brush 401 includes a handle 406 configured, for example, to be held by the hand of a user and a plurality of bristles 402. In an embodiment, the peroxide-decomposing catalyst 416 is coupled to a surface of the plurality of bristles 402, as described further herein. In another embodiment, the plurality of bristles 402 includes a crosslinked gel in which the peroxide-decomposition catalyst 416 is disposed. In this regard, oxygen is generated when a skincare formulation (not shown, see for example FIGS. 2, 3, and 6) including a peroxide as described herein is contacted with the plurality of bristles 402. Further, the applicator brush 401 is suitable to apply the skincare formulation now containing oxygen dissolved or otherwise dispersed therein to a portion of skin, thereby providing oxygen to the portion of skin.

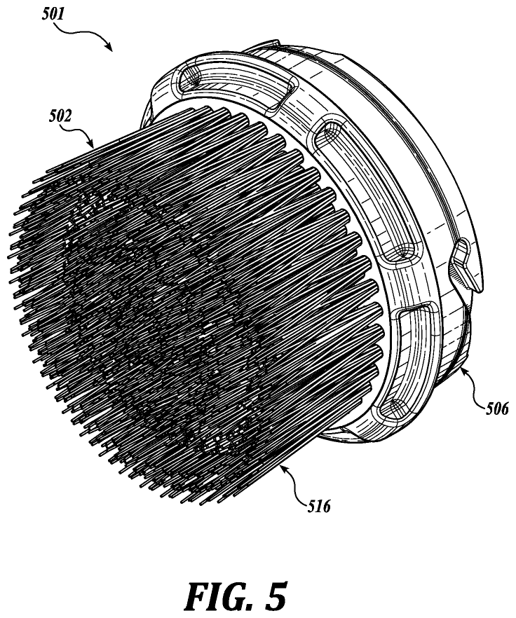

In an embodiment, the applicators of the present disclosure are in the form of an end effector, such as a brush, coupleable to an appliance and configured to receive motion from the appliance. In that regard, attention is directed to FIG. 5 in which an applicator 501 in accordance with an embodiment of the disclosure is illustrated. As shown, end effector 501 includes a base portion 506 and a plurality of bristles 502 coupled to the base portion 506. While end effector 501 including the plurality of bristles 502 is shown, it will be understood that end effectors including other contact members, such as exfoliators, applicators, and the like, are encompassed by the present disclosure. Base portion 506 is coupleable to an appliance (not shown) and to receive motion from the appliance, such as from a motor disposed in the appliance. In an embodiment, the peroxide-decomposing catalyst 516 is coupled to a surface of the plurality of bristles 502, as described further herein. In another embodiment, the plurality of bristles 502 includes a crosslinked gel in which the peroxide-decomposing catalyst 516 is disposed. In this regard, oxygen is generated when a skincare formulation (not shown, see for example FIGS. 2, 3, and 6) including a peroxide as described herein is contacted with the plurality of bristles 502. Further, the skincare formulation now containing oxygen dissolved or otherwise dispersed therein may be applied to a portion of skin with end effector 501, such as when coupled to and receiving motion from an appliance.

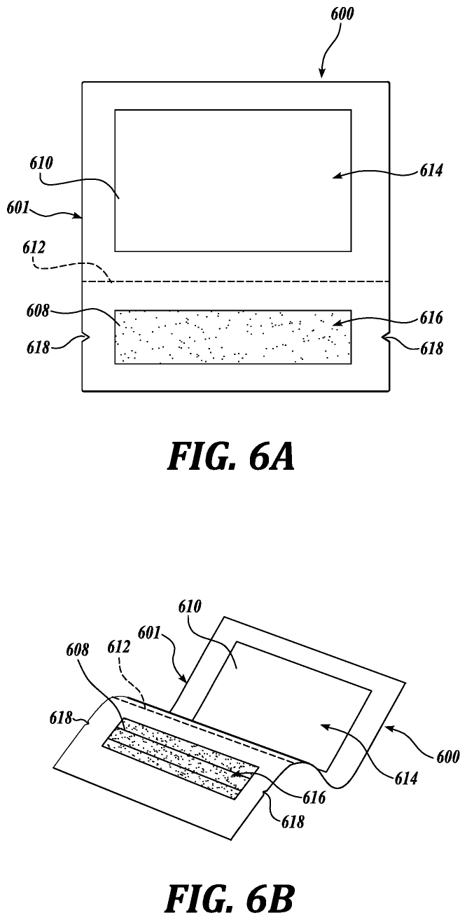

In an embodiment, the systems of the present disclosure include a sachet in which the skincare formulation and peroxide-decomposing catalyst are disposed, wherein the skincare formulation and the peroxide-decomposing catalyst are separated by a breakable barrier. In that regard, attention is directed to FIGS. 6A and 6B in which a system 600 in accordance with embodiments of the present disclosure is illustrated. FIG. 6A is a top-down plan view of system 600. FIG. 6B is a perspective view of the system 600 of FIG. 6A. As shown, system 600 is in the form of a sachet (or other container) 601 including a first compartment (also referred to as a pouch) 610 in which the skincare formulation 614 including a peroxide is disposed and a second compartment (also referred to as a pouch) 608 in which the peroxide-decomposing catalyst 616 is disposed. The first pouch 610 and the second pouch 608 are separated by a breakable barrier 612. Although the barrier 612 is described as the breakable barrier, in some embodiments, the barrier 612 is a removable or a dissolvable barrier. In an initial state in which breakable barrier 612 is intact, the first pouch 610 and the second pouch 608 are separated. When breakable barrier 612 is intact, skincare formulation 614 and peroxide-decomposing catalyst 616 are kept apart. In this regard, skincare formulation 614 and peroxide-decomposing catalyst 616 do not generate oxygen during shipment or storage that may dissipate prior to breaking the breakable barrier 612. Rather, skincare formulation 614 and peroxide-decomposing catalyst 616 are in contact and thus generate oxygen in the skincare formulation 614 when breakable barrier 612 is broken or removed, such as by a user. Accordingly, system 600 is configured to provide skincare formulation 614 having oxygen concentrations that are generally higher when applied to a portion of skin than if skincare formulation 614 were, for example, shipped in contact with peroxide-decomposing catalyst 616.

After skincare formulation 614 has been in contact with peroxide-decomposing catalyst 616 for a time sufficient to generate oxygen in the skincare formulation 614, system 600 may be used to apply skincare formulation 614 to a portion of skin. In the illustrated embodiment, sachet 601 includes notches 618 disposed at an outer edge of sachet 601 and adjacent to second pouch 608. In this regard, a user may tear open second pouch 608, for example, beginning at notches 618 when skincare formulation 614 has been in contact with the peroxide-decomposing catalyst 616 for a time sufficient to generate oxygen, so that the skincare formulation 614 and oxygen dissolved or dispersed therein may be applied to a portion of skin.

The systems of the present disclosure include skincare formulations including a peroxide dissolved or otherwise dispersed therein. As discussed further herein, the peroxides present in the skincare formulations react with the peroxide-decomposing catalysts of the applicators described further herein to decompose and provide oxygen. The peroxide can be any peroxide suitable to decompose when contacted with a catalyst to provide oxygen in the skincare formulation. In an embodiment, the peroxide is selected from the group consisting of hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof.

In an embodiment, a concentration of the peroxide in the skincare formulation is in a range of about 0.1 wt. % and about 4 wt. %. In an embodiment, the concentration of the peroxide in the skincare formulation is in a range of about 0.2 wt. % and about 2 wt. %. In an embodiment, the concentration of the peroxide in the skincare formulation is in a range of about 0.2 wt. % and about 1 wt. %. Such concentrations are generally sufficient to provide high levels of oxygen in the skincare formulations after contact with the peroxide-decomposing catalyst and provide the benefits of oxygen therapy, as discussed further herein.

The skincare formulations described herein include any skincare formulation including a peroxide. Such skincare formulations can include, for example, gels, pastes, oil/water emulsions, water/oil emulsions, foams, and the like. In an embodiment, the skincare formulation is selected from the group consisting of a moisturizer, an exfoliant, a cleaning formulation, a sunscreen, an antibiotic ointment, and combinations thereof.

In an embodiment, the skincare formulation further includes one or more additives. In an embodiment, the additive is selected from the group consisting of a clay, an alkaline agent, a humectant, an antioxidant, and combinations thereof. In certain embodiments, the clay and alkaline agents each accelerate decomposition reactions of the peroxide, thus providing increased amounts of oxygen in skincare formulations contacted by a peroxide-decomposing catalyst. In an embodiment, the clay includes zeolites. Without wishing to be bound by theory, it is believed that the zeolites accelerate decomposition reactions between peroxide-decomposing catalysts and the peroxides. In an embodiment, the alkaline agent is selected from the group consisting of alkali metal hydroxides (e.g., lithium hydroxide, sodium hydroxide, potassium hydroxide, and magnesium hydroxide), alkaline-earth metal hydroxides (e.g., calcium hydroxide), alkali metal silicates, (e.g., sodium silicate, lithium silicate, potassium silicate, magnesium silicate, aluminum silicate, and fluorphlogopite), alkali metal carbonates (e.g., lithium carbonate, sodium carbonate, potassium carbonate, and sodium bicarbonate), alkaline-earth metal carbonates (e.g., calcium carbonate), organic carbonates (e.g., guanidine carbonate), basic amino acids (arginine, lysine, and histidine), polymeric forms of basic amino acids (e.g. poly(arginine) and poly(lysine)), organic amines such as alkanolamines (e.g., monoethanolamine, diethanolamine, triethanolamine, and aminomethyl propanol), ammonium hydroxide, and combinations thereof.

Humectants and antioxidants are typically beneficial to the skin of a user. Furthermore, antioxidants are typically suitable to decrease an amount of reactive oxygen species (ROS), such as in the skincare formulation. In certain embodiments, the oxygen provided by decomposed peroxides generates ROS, which may be harmful to users. By decreasing the amount of ROS present in the skincare formulation with antioxidants a health benefit may be conferred to the user in addition to the benefits associated with oxygen therapy described further herein.

In another aspect, the present disclosure provides a method of contacting a portion of skin with oxygen gas. In an embodiment, the method includes contacting a skincare formulation including a peroxide with an applicator including a peroxide-decomposing catalyst, thereby generating oxygen gas in the skincare formulation; and contacting the portion of skin with the skincare formulation with the applicator.

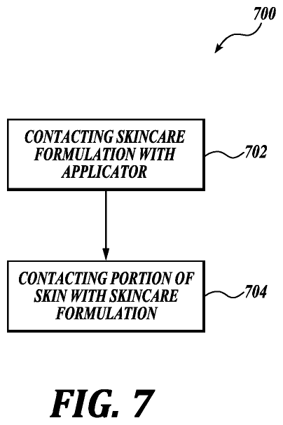

In that regard, attention is directed to FIG. 7, in which a method 700 according to embodiments of the present disclosure is schematically illustrated.

Method 700 may begin with process block 702, which includes contacting the skincare formulation with an applicator. In an embodiment, contacting the skincare formulation with an applicator includes contacting the skincare formulation with an applicator as described further herein, such as with respect to FIGS. 1-6B. In an embodiment, the applicator is selected from applicators 101, 201, 301, 401, 501, and 601. In some embodiments, peroxides in the skincare formulation decompose only when in contact with the peroxide-decomposing catalyst. In this regard, applying the skincare formulation to a portion of skin with the applicator, such as with a brush, end effector, rolling applicator, spatula, and the like, for an extended period of time provides greater oxygen levels in the skincare formulation.

In an embodiment, contacting the skincare formulation with the applicator includes contacting the skincare formulation with the applicator for a time sufficient to decompose the peroxide in the skincare formulation thereby generating oxygen. In an embodiment, the time sufficient to decompose the peroxide in the skincare formulation thereby generating oxygen is a time in a range between 30 seconds and 20 minutes. In an embodiment, the time sufficient to decompose the peroxide in the skincare formulation thereby generating oxygen is a time in a range between 1 minute and 10 minutes. In an embodiment, the time sufficient to decompose the peroxide in the skincare formulation thereby generating oxygen is a time in a range between 1 minute and 5 minutes.

In an embodiment, an oxygen concentration in the skincare formulation 1 minute after contacting the skincare formulation with the peroxide-decomposing catalyst is in a range of about 15 mg/L to about 100 mg/L. In an embodiment, an oxygen concentration in the skincare formulation 1 minute after contacting the skincare formulation with the peroxide-decomposing catalyst is in a range of about 20 mg/L to about 75 mg/L. In an embodiment, an oxygen concentration in the skincare formulation 1 minute after contacting the skincare formulation with the peroxide-decomposing catalyst is in a range of about 30 mg/L to about 50 mg/L.

In an embodiment, a concentration of the peroxide in the skincare formulation is in a range of about 0.1 wt. % and about 4 wt. %. In an embodiment, the concentration of the peroxide in the skincare formulation is in a range of about 0.2 wt. % and about 2 wt. %. In an embodiment, the concentration of the peroxide in the skincare formulation is in a range of about 0.2 wt. % and about 1 wt. %.

In an embodiment, the skincare formulation is any skincare formulation described herein. In an embodiment, the skincare formulation includes an additive selected from the group consisting of a clay, an alkaline agent, a humectant, an antioxidant, and combinations thereof. In an embodiment, the peroxide is selected from the group consisting of hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof.

In an embodiment, process block 702 is followed by process block 704, which includes contacting a portion of skin with skincare formulation. As described further herein, by contacting the skincare formulation containing a peroxide with the applicators described herein including a peroxide-decomposing catalyst, oxygen is generated in the skincare formulation. In an embodiment, the skincare formulation is contacted by the applicator prior to the skincare formulation contacting the portion of skin. In an embodiment, the skincare formulation contacts the applicator and the portion of skin simultaneously. In an embodiment, the skincare formulation is contacted with the portion of skin prior to contacting the skincare formulation with the applicator. In any event, by contacting the portion of skin with the skincare formulation and contacting the skincare formulation with the applicator, the portion of skin is exposed to the oxygen in the skincare formulation, thereby providing the portion of skin with the benefits of oxygen therapy discussed further herein.

The order in which some or all of the process blocks appear in method 700 should not be deemed limiting. Rather, one of ordinary skill in the art having the benefit of the present disclosure will understand that some of the process blocks may be executed in a variety of orders not illustrated, or even in parallel. As above, in some embodiments, process block 702 precedes process block 704. In some other embodiments, process block 704 precedes process block 702.

In accordance with the preceding embodiment, provided is a system comprising: a skincare formulation comprising a peroxide; and an applicator that carries a peroxide-decomposing catalyst, wherein the peroxide-decomposing catalyst is configured to come in contact with the peroxide and decompose the peroxide to provide oxygen gas proximate to a skin of a user.

In accordance with some embodiments, the peroxide-decomposing catalyst is coupled to an application surface of the applicator.

In accordance with some embodiments, the application surface is a surface of a mask configured to conform to a face of a wearer.

In accordance with some embodiments, the application surface is selected from the group consisting of a bristle surface of a brush, a roller surface of a rolling applicator, and a blade surface of a spatula.

In accordance with some embodiments, the skincare formulation is disposed in a first compartment of a sachet and the application surface is a surface of a second compartment of the sachet, and wherein the first compartment and the second compartment are fluidically separated by a breakable barrier.

In accordance with some embodiments, the peroxide-decomposing catalyst is coupled to the application surface with a binder.

In accordance with some embodiments, the binder is selected from the group consisting of latexes, polyacrylates, elastomers, hydrogels, and combinations thereof.

In accordance with some embodiments, the applicator includes a crosslinked hydrogel including the peroxide catalyst dispersed therein.

In accordance with some embodiments, the crosslinked hydrogel is freestanding.

In accordance with some embodiments, the crosslinked hydrogel is in a shape of a mask configured to conform to a face of the user.

In accordance with some embodiments, the crosslinked hydrogel is in a shape of one or more bristles of a brush.

In accordance with some embodiments, the peroxide-decomposing catalyst is selected from the group consisting of iron oxide and a transition metal-based catalyst; an iron salt; sodium tungstate dehydrate, phosphoric acid, and a phase-transfer catalyst comprising a quaternary ammonium hydrogen sulfate; manganese dioxide; sodium bicarbonate and manganese dioxide; and a manganese salts.

In accordance with some embodiments, the skincare formulation further comprises an additive selected from the group consisting of a clay, an alkaline agent, a humectant, an antioxidant, and combinations thereof.

In accordance with some embodiments, the peroxide is selected from the group consisting of hydrogen peroxide, urea peroxide, carbamide peroxide, calcium peroxide, magnesium peroxide, barium peroxide, zinc peroxide, lithium peroxide, dibenzoyl peroxide, peracetic acid, sodium peracetate, and combinations thereof.

In accordance with some embodiments, a concentration of the peroxide in the skincare formulation is between about 0.1 wt. % and about 4 wt. %.

In accordance with some embodiments, the skincare formulation is selected from the group consisting of an emulsion, a paste, a gel, and combinations thereof.

In accordance with the preceding embodiment, provided is a method of contacting a portion of skin with oxygen gas comprising: contacting a skincare formulation that includes a peroxide with an applicator that includes a peroxide-decomposing catalyst, thereby generating oxygen gas in the skincare formulation; and contacting the portion of skin with the skincare formulation.

In accordance with some embodiments, the peroxide-decomposing catalyst is coupled to an application surface of the applicator, and wherein contacting the skincare formulation with the peroxide-decomposing catalyst includes contacting the skincare formulation with the application surface of the applicator.

In accordance with some embodiments, an oxygen concentration in the skincare formulation 1 minute after contacting the skincare formulation with the peroxide-decomposing catalyst is in a range of about 15 mg/L to about 100 mg/L.

In accordance with some embodiments, contacting the portion of skin with the skincare formulation includes contacting the portion of skin with the skincare formulation for a duration of between about 1 minute and about 20 minutes.

While illustrative embodiments have been illustrated and described, it will be appreciated that various changes can be made therein without departing from the spirit and scope of the invention.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.” Further, it should be noted that for purposes of this disclosure, terminology such as “upper,” “lower,” “vertical,” “horizontal,” “inwardly,” “outwardly,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings. The term “about” means plus or minus 5% of the stated value.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A system for forming a cosmetic mask, comprising:
a) a first composition comprising:
i) at least one oxidizer (peroxide); and
ii) at least one cross-linkable polymer;
b) a second composition comprising:
i) at least one alkaline booster;
ii) at least one catalyst; and
wherein contacting each of the first and second compositions together on a substrate results in cross-linking the cross-linkable polymer, whereby a continuous film is formed and simultaneously oxygen is released and contained within the film.

2. The system for forming a cosmetic mask according to claim 1, wherein one or both of the first and second compositions are aqueous.

3. The system for forming a cosmetic mask according to claim 1, wherein the at least one oxidizer comprises at least one peroxide selected from one or a combination of hydrogen peroxide, PVP peroxide, urea peroxide, carbamide peroxide, calcium peroxide, magnesium peroxide, barium peroxide, zinc peroxide, lithium peroxide, dibenzoyl peroxide, peracetic acid, and sodium peracetate.

4. The system for forming a cosmetic mask according to claim 1, wherein the at least one cross-linkable polymer comprises at least one alginate.

5. The system for forming a cosmetic mask according to claim 1, wherein the at least one alkaline booster is selected from one or a combination of guanidine carbonate and arginine.

6. The system for forming a cosmetic mask according to claim 1, wherein the at least one cross-linking catalyst is selected from one or a combination of manganese gluconate, manganese pyrrolidone carboxylic acid, zinc gluconate, zinc pyrrolidone carboxylic acid, calcium chloride, calcium pyrrolidone carboxylic acid, and calcium gluconate.

7. The system for forming a cosmetic mask according to claim 1, wherein one or both of the compositions comprises at least one fatty compound and wherein the at least one fatty compound is selected from one or a combination of mineral oil, hemisqualane, dimethicone, squalane, castor oil, isohexadecane, and coconut oil.

8. The system for forming a cosmetic mask according to claim 1, wherein the at least one oxidizer is present in the first composition from about 2% by weight, based on the total weight of the composition.

9. The system for forming a cosmetic mask according to claim 1, wherein the at least one cross-linkable polymer is present in the first composition from about 1% by weight, based on the total weight of the composition.

10. The system for forming a cosmetic mask according to claim 1, wherein the at least one fatty compound is present in the first composition from at least about 20% by weight, based on the total weight of the composition.

11. The system for forming a cosmetic mask according to claim 1, wherein the at least one alkaline booster is present in the second composition from about 2% by weight, based on the total weight of the composition.

12. The system for forming a cosmetic mask according to claim 1, wherein the at least one catalyst is present in the second composition from about 0.3% by weight, based on the total weight of the composition.

13. The system for forming a cosmetic mask according to claim 1, wherein one or both of the compositions comprises at least one fatty compound, and wherein the at least one fatty compound is present in the second composition from at least about 30% by weight, based on the total weight of the composition.

14. A method of forming an occlusive cosmetic mask for providing oxygenate brightening to skin, comprising:
a) applying to a region of skin a first composition comprising a combination of at least one oxidizer, and at least one cross-linkable polymer;
b) applying to the region of skin a second composition comprising, at least one alkaline booster, and at least one catalyst wherein one or both of the compositions optionally comprises at least one fatty compound.

15. The method of forming an occlusive cosmetic mask according to claim 14, wherein the steps of the method include (a) and (b) in any order.

16. The method of forming an occlusive cosmetic mask according to claim 14, wherein each of the compositions is aqueous and is applied in liquid form.

17. The method of forming an occlusive cosmetic mask according to claim 16, wherein one or both of the compositions is applied using a spray applicator.

18. The method of forming an occlusive cosmetic mask according to claim 14, wherein the compositions are preblended before applying to the region of skin.

19. An article of manufacture for providing a skin brightening system in an occlusive continuous film, comprising: at least first and second separately contained compositions, the brightening system comprising hydrogen peroxide; at least one cross-linkable polymer; at least one alkaline booster; at least one catalyst; and optionally at least one fatty compound which when present is from about at least 30% by weight, based upon the total weight of the composition, wherein the skin-brightening cosmetic system is ammonia-free and persulfate-free;
a) the first composition comprising the hydrogen peroxide, and the cross-linkable polymer, and
b) the second composition comprising the alkaline booster, and the catalyst
wherein either one or both of the first and second compositions comprises one or more of the optional fatty compounds, if present, and optionally one or more of solvents, and optionally one or more actives and other components.

20. The article of manufacture according to claim 19, wherein the first and second compositions are one of (i) contained to be mixable prior to application to skin, and (ii) contained to be applied separately, in any order.
